(19) United States
(12) Patent Application Publication
(10) Pub. No.: US 2021/0317194 A1
(43) Pub. Date: Oct. 14, 2021

(54) ANTIBODY FOR HAIR IMPROVEMENT USE

(71) Applicant: OSTRICH PHARMA KK, Kyoto (JP)

(72) Inventor: Yasuhiro TSUKAMOTO, Osaka (JP)

(21) Appl. No.: 17/285,605

(22) PCT Filed: Oct. 16, 2019
(86) PCT No.: PCT/JP2019/040681

§ 371 Date: Apr. 15, 2021

(30) Foreign Application Priority Data
Oct. 17, 2018 (JP) 2018-195862

(51) Int. Cl. C07K 16/12; C07K 16/02; A61Q 7/00; A61K 8/64

(52) U.S. Cl. CPC C07K 16/1267; C07K 16/1271; C07K 16/02; C07K 2317/11; A61K 8/64; C07K 2317/23; A61Q 7/00

(57) ABSTRACT

The present disclosure provides a composition and a method both for improving the hair. In one embodiment, the hair is improved using an antibody. In one embodiment, the antibody is produced using a bacterium occurring on the scalp or a hair root as an immunogen. In one aspect, the present invention also provides a method for improving the hair in a subject, including the step of administering an antibody produced using a microorganism-derived substance as an immunogen to the subject. The antibody according to the present invention may be administered in the form of a composition. According to the present invention, the promotion of hair growth or the prevention of hair loss can be achieved.

### TECHNICAL FIELD

The present invention relates to an antibody-containing composition for improvement in hair and a method for improving hair using an antibody.

### BACKGROUND ART

A skin is an organ covering the surface of a body and has a layered structure including epidermis, dermis, and subcutaneous tissue. A skin is responsible for the function of sensing of external stimuli, protection from external stimuli, adjustment of exchange of substances (e.g., water) with the external environment and the like. Further, a mucous membrane is also an organ covering the surface of a body and adjusts the absorption and secretion of substances.

Ointment, cream, lotion or the like are used as a preparation for external use to be applied to the body surface. However, it is considered that since it is commonly difficult for molecules with a high molecular weight such as proteins to permeate inside the body from the skin or the mucous membrane, it is also difficult for those molecules to be applied to the body surface to exhibit their function inside the body.

Further, a skin has hair. Many aspects of the control mechanism relating to hair, e.g., the alopecia-causing mechanism, are not understood. In addition, a ground-breaking therapeutic method has not been discovered up to this point. Several substances for hair growth promotion have been proposed, including 3′-phosphoadenosine-5′-phosphosulfate disclosed in International Publication No. WO 2012/057336.

### CITATION LIST

### Patent Literature

[PTL 1] International Publication No. WO 2012/057336

### SUMMARY OF INVENTION

### Solution to Problem

The present inventor unexpectedly discovered that an antibody is effective for improvement in hair and completed the present invention. Surprisingly, administration of an antibody of which immunogen is a substance that is not present in a subject to the subject exhibited a hair growth promotion and/or hair loss suppression effect as demonstrated in the Examples.

Thus, the present invention provides the following.

### (Item 1)

A composition for improvement in hair in a subject, comprising an antibody of which immunogen is a substance that is not present in the subject.

### (Item 2)

The composition of item 1, wherein the substance that is not present in the subject is a microorganism-derived product or an allergy substance.

### (Item 3)

The composition of item 1 or 2, wherein the improvement in hair includes hair growth promotion or hair loss suppression.

### (Item 4)

The composition of any one of items 1 to 3, wherein the composition is for application to a skin.

### (Item 5)

The composition of item 4, wherein the skin is a scalp.

### (Item 6)

The composition of any one of items 1 to 5, wherein the microorganism-derived product includes a microbial body, a homogenate, a toxin, a cytoplasm, a cell wall, or any combination thereof.

### (Item 7)

The composition of any one of items 1 to 6, wherein the microorganism includes a microorganism that is present on a scalp or a hair root.

### (Item 8)

The composition of item 7, wherein the microorganism includes a bacterium.

### (Item 9)

The composition of item 9, wherein the bacterium includes a bacterium of the genus Staphylococcus or the genus Propionibacterium.

### (Item 10)

The composition of item 9, wherein the bacterium includes Staphylococcus aureus or Propionibacterium acnes.

### (Item 11)

The composition of any one of items 1 to 10, wherein the antibody is derived from ayes.

### (Item 12)

The composition of item 11, wherein the aves are ostriches.

### (Item 13)

The composition of any one of items 1 to 12, wherein the composition is in the form of shampoo, conditioner, wax for hair, or cream for skin.

### (Item 14)

The composition of any one of items 1 to 13, wherein the composition is a cosmetic product.

### (Item 15)

The composition of any one of items 1 to 14, wherein the composition is for women.

### (Item 16)

The composition of any one of items 1 to 15, wherein the composition is for men.

In the present invention, it is intended that the above one or more characteristics can be further combined to be provided in addition to the explicitly described combinations. Those skilled in the art will appreciate still further embodiments and advantages of the present invention by reading and understanding the detailed description below as required.

### Advantageous Effects of Invention

The antibody of the present invention of which immunogen is a substance that is not present in a subject can be effective for improvement in hair. In particular, the antibody of the present invention can be useful for hair loss suppression and/or hair growth promotion.

### BRIEF DESCRIPTION OF DRAWINGS

FIG. 1A shows an evaluation criterion of the male hair state according to the Norwood scale.

FIG. 1B shows an evaluation criterion of the female hair state according to the Ludwig scale.

FIG. 2A shows observation images of a patient with androgenetic alopecia (AGA) who was determined to be the Norwood Type III prior to the treatment with the antibody of the present invention (left) and in month 3 from the treatment (right).

FIG. 2B shows observation images of a woman who was determined to be the Ludwig Type II prior to the treatment with the antibody of the present invention (left) and in month 3 from the treatment (right).

FIG. 2C shows the result of counting the number of hair roots per area of ten men at each point of time prior to the treatment with the antibody of the present invention (left) and in month 3 from the treatment (right). The vertical axis indicates the number of hair roots per 1 cm², while the horizontal axis indicates the hair state evaluation according to the Norwood scale of each man prior to the treatment with the antibody.

FIG. 2D shows the result of counting the number of hair roots per area of four women at each point of time prior to the treatment with the antibody of the present invention (left) and in month 3 from the treatment (right). The vertical axis indicates the number of hair roots per 1 cm², while the horizontal axis indicates the hair state evaluation according to the Ludwig scale of each woman prior to the treatment with the antibody.

### DESCRIPTION OF EMBODIMENTS

The present invention is explained hereinafter with reference to the best embodiments. Throughout the entire present specification, a singular expression should be understood as encompassing the concept thereof in the plural form, unless specifically noted otherwise. Thus, singular articles (e.g., “a”, “an”, “the”, and the like in the case of English) should also be understood as encompassing the concept thereof in the plural form, unless specifically noted otherwise. Further, the terms used herein should be understood as being used in the meaning that is commonly used in the art, unless specifically noted otherwise. Thus, unless defined otherwise, all terminologies and scientific technical terms that are used herein have the same meaning as the general understanding of those skilled in the art to which the present invention pertains. In case of a contradiction, the present specification (including the definitions) takes precedence.

The definitions and/or basic technical contents of the terms particularly used herein are appropriately explained hereinafter.

### (Definition)

As used herein, “or” is used when “at least one or more” of the matters listed in a sentence can be employed. The same applies to “or”. As used herein, when “within the range” of “two values” is explicitly described, the range also encompasses the two values themselves.

As used herein, “about” refers to variation up to ±10% from an indicated value being allowed.

As used herein, “subject” means any human or non-human animal. The non-human animal includes, for example, dogs, cats, mice, rabbits, horses, pigs, monkeys, chimpanzees and the like. In one embodiment, the non-human animal can be a pet animal or an ornamental animal. As used herein, a substance that is not present in a subject refers to a substance that is not usually present in the body (e.g., inside the skin or the mucous membrane) of the subject. For example, a substance that is not present in a human can be a component of a microorganism present on the human epidermis, a component of a microorganism present in the human intestine, and a component of a virus infecting a human or a parasite parasitic on a human, or the like.

As used herein, “improvement in hair” refers to any desired effect in body hair. In one embodiment, the improvement in hair includes, but is not limited to, for example, hair growth promotion, hair loss suppression, prevention and/or therapy of alopecia, increase in body hair, increase in the growth speed of body hair, increase in the body hair density, uniformized overall appearance of body hair and the like.

As used herein, “immunogen” refers to a target substance, an antibody binding to which is intended to be obtained. An immunogen can be, for example, a cell, an intracellular component, an extracellular component, a cell membrane, a cell wall, a secretion of an organism, or an allergy substance or the like. As used herein, an immunogen may be purified or unpurified.

It is understood that as used herein, “medicament” is interpreted in the broadest sense in the art, encompasses any drug, is used as a concept encompassing not only drugs applied to humans but also drugs applied to animals (veterinary medicament) as well as pharmaceutical products, quasi drugs and the like under the Pharmaceutical Act, and encompasses agents, compositions and the like of any use intended for improvement, therapy, or prevention of a disease, a disorder or a condition in need thereof. Such examples include application in the cosmetic field, the health care field, the medical field, the veterinary science or the like. A medicament normally can contain a solid, liquid, emulsion, or foam excipient and contain, as required, an additive such as stabilizer, pH regulator, surfactant, emulsifying agent, moisturizing agent, buffer, disintegrating agent, flavoring agent, delayed release agent, lubricant, binder or colorant. The form of a pharmaceutical product includes, but is not limited to, injections, patch agents, microneedles, agents for external use (e.g., ointments, creams, lotions, compresses, sprays), suppositories, tablets, capsules, granules, powders, fine granules, sustained release formulations and the like. The component, the antibody, the compound, the composition or the like of the present invention can be prepared as a pharmaceutical composition in combination with a component such as a pharmaceutically acceptable common carrier or excipient.

As used herein, “cosmetic product” refers to any product intended to be used by rubbing or spraying over the body or by other methods similar thereto in order to clean the body, beautify the body, increase the attractiveness, change the appearance, or keep the skin or hair healthy. As used herein, “cosmetic product” is not limited to the “cosmetic product” pursuant to the so called Act on Pharmaceuticals and Medical Devices (former Pharmaceutical Act), but may be any of, for example, quasi drugs, pharmaceutical products, and general goods.

### (Antibody)

In one aspect, the antibody of the present invention can be any antibody of which immunogen is a substance that is not present in a subject. As used herein, an antibody obtained by using a certain substance as an immunogen encompasses an antibody produced when the substance is administered to an animal having an immune system, an antibody produced from cells binding to the substance among cells expressing an antibody recognition site on the cell surface, and an antibody produced by modifying these antibodies without changing the antigen binding. For example, an antibody modified in this manner includes, but is not limited to, an antibody modified without changing a CDR and an antibody in which only the constant domain has been modified. Those skilled in the art can make such an antibody using any well-known technique. Further, as long as a substance is identified as an immunogen, those skilled in the art can readily identify which antibody is an antibody obtained by using this substance as an immunogen.

In one embodiment, the antibody of the present invention can be antibodies obtained by using a microorganism-derived product or an allergy substance as an immunogen. In one embodiment, the antibody of the present invention is an antibody produced from organisms including aves, microorganisms, mammals, insects and the like. In one embodiment, the antibody of the present invention can be antibodies (e.g., IgY) obtained by inoculating female aves (e.g., chickens, ostriches, emus, quails, ducks, turkeys, geese) with an immunogen and collecting the antigen from the egg yolk. In one embodiment, the antibody of the present invention can be derived from ostriches. In one embodiment, the antibody of the present invention may be an antibody produced by further modifying an antibody produced from organisms.

In one embodiment, the antibody of the present invention may be polyclonal antibodies, wherein the polyclonal antibodies may be antibodies obtained by using a single substance (e.g., single species of microorganism, single species of protein, or the like) as an immunogen, or may be antibodies obtained by using a plurality of substances (e.g., a plurality of species of microorganism, a plurality of species of protein in a single species of microorganism, or the like) as an immunogen. In one embodiment, the antibody of the present invention may be a specific one or plurality of species of antibody which was isolated, purified or concentrated. In one embodiment, the antibody of the present invention or at least one type of the antibody of the present invention binds to or specifically binds to an immunogen used when obtaining said antibody or a component thereof.

An antibody against a desired immunogen can be obtained from female aves using a known method. Upon immunization, any additive component such as adjuvant, salt and stabilizer can be utilized with an immunogen. Further, upon immunization, an immunogen can be administered under any optimal condition (e.g., dosage, administration site, pH, temperature or the like). Regarding immunization, booster may be performed after priming.

For example, immunization of an ostrich is typically performed in, but not limited to, the following manner.
Priming: An immunogen is mixed with an appropriate adjuvant such as Freund's Complete Adjuvant, and the mixture is inoculated into the muscle of the loin of a female ostrich.
Booster: After priming, booster is performed three times every other week. An immunogen is mixed with an appropriate adjuvant such as Freund's Incomplete Adjuvant, and the mixture is inoculated into the muscle of the loin of the female ostrich.

Antibodies are purified from an ostrich egg laid after week 2 of the booster.

The microorganism which is used as an immunogen in the present invention can be a microorganism present on a scalp or a hair root, which includes bacteria, viruses, fungi, worms, insects and the like (e.g., Staphylococcus aureus group, Streptococcus, Pseudomonas aeruginosa, Micrococcus, Candida, Aspergillus, Trichophyton, mites, Malassezia or the like). In one embodiment, the microorganism which is used as an immunogen can include a bacterium of the genus Staphylococcus and/or the genus Propionibacterium. In one embodiment, the microorganism which is used as an immunogen can include Staphylococcus aureus and/or Propionibacterium acnes. In one embodiment, the allergy substance which is used as an immunogen in the present invention includes house dust, allergens of cats or dogs and the like.

The microorganism-derived product which is used in the present invention may be a microorganism itself, a substance contained in or secreted from a microorganism, a degradation product of these substances, or a substance produced/formed by interaction between these substances and a component in a subject. Typically, the microorganism-derived product can be an unprocessed microbial body, a homogenate, a toxin, an enzyme, a polysaccharide or a pigment produced by a bacterium, a cell wall or a cytoplasm, a spore, a flagellum or a fimbria of a bacterium, or a part thereof, and can be preferably a homogenate or a toxin of a bacterium. The microorganism-derived product as an immunogen is preferably a homogenate. The microorganism-derived product as an immunogen can be preferably a toxin produced by a bacterium, e.g., an endotoxin, an exotoxin, a capsule, a spore. For example, the component of the microorganism includes enterotoxins, TSST-1, coagulase, protease, lipase, protein A, pyocyanin, pyoverdine, pyorubin, exotoxin A, hemolysin, alkaline peptidase, elastase, exoenzyme S, rhamnolipid, mold toxin: mycotoxin), and the like. The antibody of the present invention or at least one species of the antibody of the present invention can bind or specifically bind to any of these components. For improving hair, an antibody obtained by using a homogenate of a microorganism (e.g., bacteria) as an immunogen can be preferred. Although not intending to be bound by any theory, an antibody obtained by using a homogenate of a microorganism as an immunogen can exhibit an especially excellent effect by acting on not only the microorganism itself but also other microorganism-derived products.

Although not intending to be bound by any theory, it is considered that the antibody of the present invention exerts an effect by the following mechanism. Specifically, indigenous bacteria (normal bacterial flora) present on the scalp skin are disrupted by daily shampooing or hair washing. The nutrient of indigenous bacteria is often sebum, so that the indigenous bacteria will lose their balance when the sebum of hair roots is degenerated due to Propionibacterium acnes or the like. It is considered that, in such a state, pathogenic bacteria including Staphylococcus aureus and Pseudomonas aeruginosa or fungi easily proliferate, whose microbial body, toxin, enzyme or the like induces inflammation in hair roots or damage to hair root cells that causes hair loss, resulting in inhibition of hair growth. Dripping of an antibody against those bacteria or fungi onto the scalp causes an antigen-antibody reaction which neutralizes hair loss factors and hair growth inhibiting factors. As a result, hair growth is promoted.

In a preferred embodiment, the antibody used in the composition of the present invention can be antibodies obtained by immunizing aves with a homogenate of Staphylococcus aureus and a homogenate of Propionibacterium acnes as an immunogen. In a particularly preferred embodiment, the antibody used in the composition of the present invention can be antibodies obtained by immunizing an ostrich with a homogenate of Staphylococcus aureus and a homogenate of Propionibacterium acnes as an immunogen.

In one embodiment of the present invention, an immunogen can be obtained by homogenizing a microbial body or an allergen in phosphate buffer solution (PBS) by a homogenizer. Homogenization can be performed at pH about 6 to about 8, preferably at pH about 7 under refrigeration (4 degrees). A high speed rotation machine for extracting protein can be used for a homogenizer. Preferably, the liquid is directly used as an immunogen to immunize aves without centrifuging the liquid after homogenization. Although a protease inhibitor or a stabilizer is not used, trehalose may be added so as to be 4% of the liquid. Furthermore, the liquid obtained in this manner may be mixed with an adjuvant for immunization, but an antibody can be produced even without using an adjuvant.

### (Method)

In one aspect, the present invention also provides a method for improving hair of a subject, comprising a step of administering an antibody to the subject. In this method, the antibody of the present invention may be administered as a composition as described below. In one embodiment, the antibody used in this method can be any antibody of which immunogen is a substance that is not present in a subject. In one embodiment, the antibody used in this method can be antibodies obtained by using a microorganism-derived product or an allergy substance as an immunogen.

The antibody of the present invention can be administered to a mammal, preferably a human, in any pharmaceutically acceptable administration form as a bolus or through continuous infusion over a certain period via a skin surface (e.g., scalp surface) route, a transdermal route, a rectal route, an intramuscular route, an intraperitoneal route, an intracerebral route, a subcutaneous route, an intraarticular route, an intrasynovial route, an intrathecal route, an oral route, a topical route or an inhalation route. The present invention is particularly useful for hair growth promotion or hair loss suppression, and administration to the skin surface (e.g., scalp surface) is particularly preferred for this purpose. Although it is considered that antibodies usually do not permeate the skin, the present inventor surprisingly found that administration of an antibody of which immunogen is a substance that is not present in a human to the skin surface results in exhibition of an effect of improving hair based on the mechanism in a human body. Further, it is expected that topical application to the skin surface has fewer side effects as compared to hair growth agents such as minoxidil that is orally administered.

An appropriate dosage of the antibody of the present invention can depend on the type and degree of the hair improvement effect of interest, the state and progress of hair, the application in the past, the medical history and response to the antibody of a subject, and the discretion of an attending physician. The antibody of the present invention can be appropriately administered to a subject at one time or over a series of treatments.

The antibody of the present invention may be administered in combination with agents that protect the skin, agents that activate hair root function or agents that suppress microorganisms (e.g., antibiotics). The agents that suppress microorganisms include, but are not limited to, β lactam based (penicillin based, cephem based), aminoglycoside based, macrolide based, tetracycline based, quinolone based, clindamycin based, gentamicin based, chloramphenicol based, mitomycin based, vancomycin based, methicillin based, nystatin based, carbapenem based, amphotericin based, polyether based, peptide based antibiotics, sulfonamide based, nitrofuran based, isonicotinic acid hydrazide based, pyrazinamide based, arsenic compound based, antimony compound based, quinoline compound based chemotherapeutic drugs and the like. The agents that activate hair root function include minoxidil, adenosine-related compound, 3′-phosphoadenosine-5′-phosphosulfate and the like. Use of the antibody of the present invention in combination with these agents can achieve a higher level of hair growth action.

The antibody of the present invention can be useful for not only suppression of a microorganism that is used as an immunogen but also a resistant microorganism that is derived from said microorganism and acquired resistance to treatment (such as irradiation) and agents for killing the microorganism. There are some mechanisms of resistance acquisition, but typical mechanisms include acquisition of the ability of producing an agent decomposing enzyme or modifying enzyme, mutation in an agent acting site, decrease in the holes through which an agent invades a microbial body, acquisition/enhancement of an agent efflux mechanism and the like. Although not intending to be bound by any theory, since it can be possible that the antibody of the present invention suppresses proliferation or function of microorganisms by a mechanism different from these resistance acquisition mechanisms, it is considered that an antibody made by using a certain microorganism as an immunogen also exerts an effect on a resistant microorganism derived from said microorganism. Alternatively, it is considered that the antibody of the present invention exerts its effect by binding to and neutralizing a protein synthesis enzyme (e.g., enzyme for proliferation), an agent decomposing enzyme or the like that is acquired and produced by a microorganism through mutation for resistance.

### (Composition)

In one aspect, the antibody of the present invention is provided as a composition comprising the antibody of the present invention.

In one embodiment, the composition of the present invention may be a cosmetic product. The cosmetic product includes, for example, cleaning cosmetic products, hair cosmetic products, skin care products, makeup cosmetic products, aromatic cosmetic products, suntan/sunscreen cosmetic products, lip cosmetic products, and bath agents.

In one embodiment, the composition of the present invention may be a medicament. The medicament includes, for example, prophylactic agents for alopecia, therapeutic agents for alopecia, hair growth agents, hair washing agents, hair styling agents and the like.

The composition of the present invention can be in any optimal form and can be in the form of, for example, soap, shampoo, conditioner, hair treatment, wax for hair, hair styling agents such as pomade, agents for external use (e.g., ointments, creams, lotions, compresses, sprays), injections, collunaria, eye drops, oral cavity sprays, washing drugs, patch agents, microneedles, suppositories, tablets, capsules, granules, powders, fine granules, sustained release formulations or the like.

The composition of the present invention may contain a solid, liquid, emulsion, or foam excipient, and can optionally contain an additive such as stabilizer, pH regulator, surfactant, emulsifying agent, moisturizing agent, buffer, disintegrating agent, flavoring agent, delayed release agent, lubricant, binder or colorant. In one embodiment, the composition of the present invention may further contain an agent that protects the skin, an agent that activates hair root function or an agent that suppresses microorganisms (e.g., antibiotics) as described above.

In one embodiment, the composition of the present invention may be provided as a composition for women, and may be added with a suitable component (e.g., isoflavone) for this purpose. In one embodiment, the composition of the present invention may be provided as a composition for men, and may be added with a suitable component (e.g., menthol) for this purpose. In a preferred embodiment, the sex of a subject does not matter to the composition of the present invention.

### EXAMPLES

Two bacteria, Staphylococcus aureus (S. aureus; NBRC102135) and Propionibacterium acnes (P. acnes; NBRC107605), were used as an immunogen to examine the hair loss suppression/hair growth promotion effect of ostrich antibodies.

Culture suspension of each of the above two bacteria was centrifuged and precipitated. Culture medium was removed, phosphate buffer solution (pH7) was added to float bacteria, and bacterial microbial bodies were homogenized with a homogenizer at 4° C. An ostrich was immunized with the resultant homogenized liquid (homogenate) using as an immunogen.

### <Immunization of Ostrich>

Priming: each of the above homogenates in an amount of 100 μg protein content was mixed with Freund's Complete Adjuvant, and the mixture was inoculated into the muscle of the loin of a female ostrich.

Booster: After priming, booster with the above two types of homogenates was performed three times every other week. The booster was performed by mixing bacterial homogenate liquid in an amount of 100 μg with Freund's Incomplete Adjuvant and inoculating the mixture into the muscle of the loin of the already immunized female ostrich.

### <Purification of Antibody>

Antibodies were purified from an ostrich egg laid after week 2 of the booster. The method for purifying the antibodies is shown below. Antibodies from the egg yolk (IgY) were purified in the following manner.

First, to the egg yolk, 5 times the amount of TBS (20 mM of Tris-HCl, 0.15 M of NaCl, 0.5% NaN₃) and the same amount of 10% dextran sulfate/TBS were added, and the mixture was stirred for 20 minutes. 1M of CaCl₂/TBS was then added in the same amount as the egg yolk, and the mixture was stirred and left standing for 12 hours. The mixture was then centrifuged for 20 minutes at 15000 rpm, and the supernatant was collected. In addition, ammonium sulfate was added such that the final concentration would be 40%, and the mixture was left standing for 12 hours at 4° C. After that, the mixture was centrifuged for 20 minutes at 15000 rpm, and precipitates were collected. Finally, the precipitates were resuspended in TBS in the same amount as the egg yolk, and dialysis was performed with TBS. IgY with a purity of 90% or more was able to be collected by the above steps. 2 to 4 g of IgY were able to be purified from one egg yolk.

### Preparation of Antibody Solution

The components of ethyl alcohol, distilled water, and antibody undiluted solution (prepared by adding 15 mg of anti-S. aureus antibodies and 15 mg of anti-P. acnes antibodies per 1 mL of phosphate buffer solution) were mixed such that the ethyl alcohol would be 20%, the distilled water would be 79%, and the antibody undiluted solution would be 1% to prepare an antibody solution.

For three months, the above-described antibody solution was applied by dripping an appropriate amount (about 1 to 10 mL) onto the affected part (scalp with thinning hair) of men (ten men) and women (four women) twice a day in the morning and in the evening and left as is. Hair washing or the like was performed as usual. The tested men have androgenetic alopecia (AGA) and were 40 years of age, while the tested women were 59 years of age.

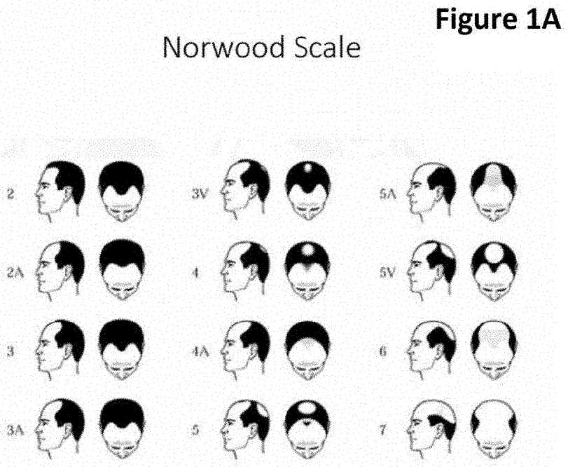

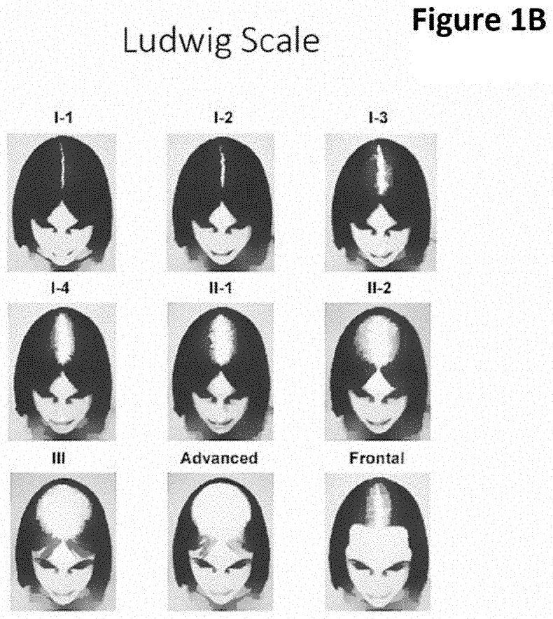

The state of the scalp was observed according to the Norwood scale (FIG. 1A) and the Ludwig scale (FIG. 1B) (Mrinal Gupta et. al., J Cutan Aesthet Surg. 2016 Jan-Mar;9(1):3-12.) to confirm a hair growth effect at two points of time, i.e., prior to the start of the use of the antibody solution and month 3. Further, the number of hairs (the number of hair roots) per unit area was counted.

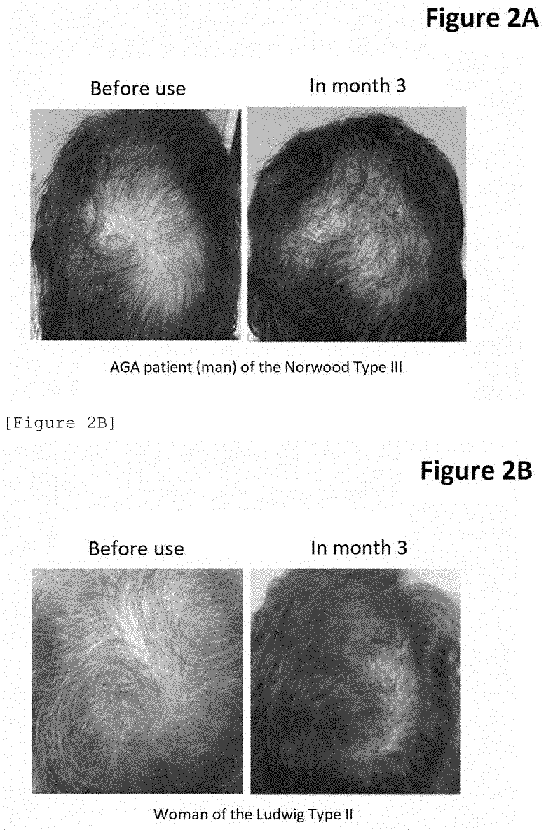

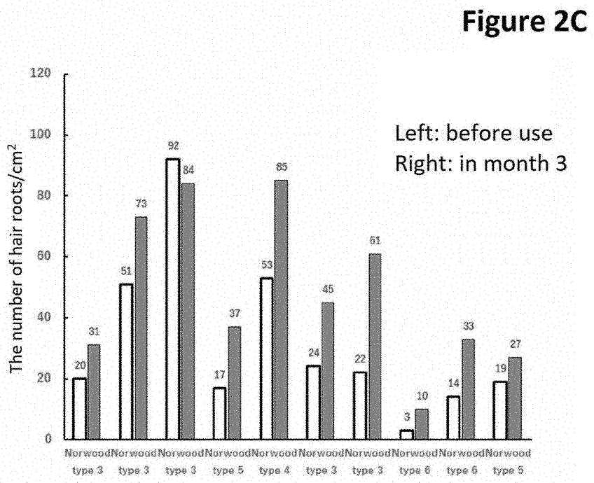

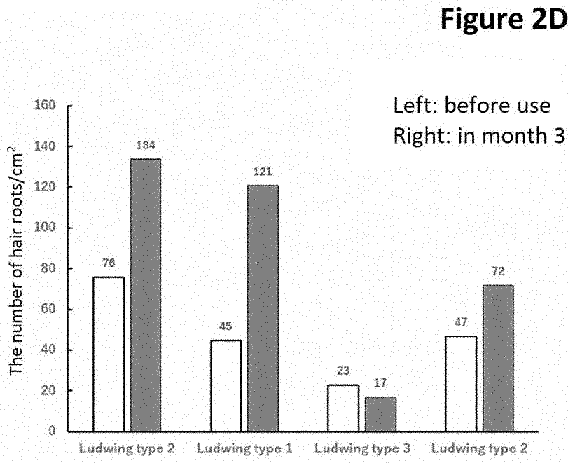

As a result, a hair growth effect was found in nine out of ten men (FIG. 2AB), while a hair growth effect was found in three out of four women (FIG. 2CD).

### Discussion

A hair growth effect was found at a high percentage in the ostrich antibody solution made by using a homogenized product of S. aureus/P. acnes as an immunogen. It has been reported that hair growth agents such as minoxidil are effective for men but ineffective on thinning hair of women due to a difference in the hair loss mechanism. However, in the present Examples, a hair growth effect was observed in both men and women. It is considered that this is because the antibodies suppressed proliferation of microorganism present on the skin, and toxins, enzymes or the like that are produced, which resulted in inactivation of substances causing thinning hair and hair loss and activation of hair roots.

Further, it has been reported that minoxidil, when orally taken, causes side effects such as decline in sexual desire, while the present Examples demonstrate that microorganisms on the scalp are specifically suppressed by direct administration of the antibodies to the skin surface, in which a hair growth effect is easily obtained. In particular, it is expected that the present invention is highly useful because the effect was found on thinning hair of women as well, for which there are not many therapeutic methods.

It is predicted that a similar hair growth promotion and/or hair loss suppression effect is exerted by antibodies made by using other microorganisms (e.g., Staphylococcus aureus group, Streptococcus, Pseudomonas aeruginosa, fungi (Micrococcus, Candida, Aspergillus, Trichophyton, Malassezia)) or allergy substances (house dust, mites, allergens of cats or dogs) as an immunogen. Further, it is predicted that a similar effect is obtained by not only ostrich antibodies but also antibodies produced in any optimal organism.

### INDUSTRIAL APPLICABILITY

A composition and a method for improvement in hair are provided by the present invention.

1. A composition for improvement in hair in a subject, comprising an antibody of which immunogen is a substance that is not present in the subject.

2. The composition of claim 1, wherein the substance that is not present in the subject is a microorganism-derived product or an allergy substance.

3. The composition of claim 1, wherein the improvement in hair includes hair growth promotion or hair loss suppression.

4. The composition of claim 1, wherein the composition is for application to a skin.

5. The composition of claim 4, wherein the skin is a scalp.

6. The composition of claim 2, wherein the microorganism-derived product includes a microbial body, a homogenate, a toxin, a cytoplasm, a cell wall, or any combination thereof.

7. The composition of claim 2, wherein the microorganism includes a microorganism that is present on a scalp or a hair root.

8. The composition of claim 7, wherein the microorganism includes a bacterium.

9. The composition of claim 8, wherein the bacterium includes a bacterium of the genus Staphylococcus or the genus Propionibacterium.

10. The composition of claim 9, wherein the bacterium includes Staphylococcus aureus or Propionibacterium acnes.

11. The composition of claim 1, wherein the antibody is derived from aves.

12. The composition of claim 11, wherein the aves are ostriches.

13. The composition of claim 1, wherein the composition is in the form of shampoo, conditioner, wax for hair, or cream for skin.

14. The composition of claim 1, wherein the composition is a cosmetic product.

15. The composition of claim 1, wherein the composition is for women.

16. The composition of claim 1, wherein the composition is for men.
